SEQUENCE LISTING (28 sequences)

SEQ ID NO: 1 - DNA, 20 bp, Artificial Sequence
  primer
cctgtagctctccaggcatc

SEQ ID NO: 2 - DNA, 20 bp, Artificial Sequence
  primer
caggcagtttcctctggaag

SEQ ID NO: 3 - DNA, 20 bp, Artificial Sequence
  primer
cacctgggtttccactcatt

SEQ ID NO: 4 - DNA, 20 bp, Artificial Sequence
  primer
ccttgtgacactgcgtgact

SEQ ID NO: 5 - DNA, 22 bp, Artificial Sequence
  primer
gttgagtgacaggaggctgctt

SEQ ID NO: 6 - DNA, 22 bp, Artificial Sequence
  primer
aggctgggaaacaaggtaggag

SEQ ID NO: 7 - DNA, 20 bp, Artificial Sequence
  primer
caatcctcttctccccaaca

SEQ ID NO: 8 - DNA, 20 bp, Artificial Sequence
  primer
tactcagcaaaccccaaagg

SEQ ID NO: 9 - DNA, 22 bp, Artificial Sequence
  primer
cctggagacttccgctactcag

SEQ ID NO: 10 - DNA, 22 bp, Homo sapiens
gatggtcagtcgggagtacagg

SEQ ID NO: 11 - DNA, 45 bp, Artificial Sequence
  primer
tacactttctttctttctttctttgcctcctgacccttccatcct

SEQ ID NO: 12 - DNA, 45 bp, Artificial Sequence
  primer
ctttctatctttctactcaataatgcctcctgacccttccatccc

SEQ ID NO: 13 - DNA, 49 bp, Artificial Sequence
  primer
cttttacaatacttcaatacaatcagacagacctcccgccctgggagag

SEQ ID NO: 14 - DNA, 49 bp, Artificial Sequence; identical to SEQ ID NO: 13
  primer

SEQ ID NO: 15 - DNA, 44 bp, Artificial Sequence
  primer
tcatcaatcaatctttttcactttacctggttcccccttttctc

SEQ ID NO: 16 - DNA, 44 bp, Artificial Sequence
  primer
ttacctttatacctttctttttacacctggttcccccttttctt

SEQ ID NO: 17 - DNA, 44 bp, Artificial Sequence
  primer
ctacaaacaaacaaacattatcaacttcccaggcaaagatgggc

SEQ ID NO: 18 - DNA, 44 bp, Artificial Sequence
  primer
tacatcaacaattcattcaatacacttcccaggcaaagatgggt

SEQ ID NO: 19 - DNA, 47 bp, Artificial Sequence
  primer
tacactttctttctttctttctttttcccccttttctyccttggtgc

SEQ ID NO: 20 - DNA, 47 bp, Artificial Sequence
  primer
cttttcatcttttcatctttcaatttcccccttttctyccttggtgt

SEQ ID NO: 21 - DNA, 45 bp, Artificial Sequence
  primer
ttacctttatacctttctttttacccatttcttctcactgccacc

SEQ ID NO: 22 - DNA, 45 bp, Artificial Sequence
  primer
tcatcaatcaatctttttcactttccatttcttctcactgccacg

SEQ ID NO: 23 - DNA, 45 bp, Artificial Sequence
  primer
atcatacatacatacaaatctacacacacactgctcttttcccca

SEQ ID NO: 24 - DNA, 45 bp, Artificial Sequence
  primer
aatccttttacattcattacttaccacacactgctcttttccccg

SEQ ID NO: 25 - DNA, 556 bp, Homo sapiens
tttccactcattctcattccctaagctaacaggcataagccagctggcaatgcacggtcccatttgttctcactgccacggaaagcatgtttatagtcttccagcagcaacgccaggtgtctaggcacagatgaacccctccttaggatccccactgctcatcatagtgcctacctttgttaaagtactagtcacgcagtgtcacaaggaatgtttacttttccaaatccccagctagaggccagggatgggtcatctatttctatatagcctgcacccagattgtaggacagagggcatgctcggtaaatatgtgttcattaactgagattaaccttccctgagttttctcacaccaaggtgaggaccatgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccag

SEQ ID NO: 26 - DNA, 511 bp, Homo sapiens
  misc_feature (256)..(256): wherein “n” is c or t
gtaggagagaaggtcttctccagcaatgggcagtccatcacttttgatgccattcaggaggcatgtgccagagcaggcggccgcattgctgtcccaaggaatccagaggaaaatgaggccattgcaagcttcgtgaagaagtacaacacatatgcctatgtaggcctgactgagggtcccagccctggagacttccgctactcagacgggacccctgtaaactacaccaactggtaccgaggggagcccgcaggtnggggaaaagagcagtgtgtggagatgtacacagatgggcagtggaatgacaggaactgcctgtactcccgactgaccatctgtgagttctgagaggcatttaggccatgggacagggaggacgctctctggccttcggcctccatcctgaggctccacttggtctgtgagatgctagaactccctttcaacagaattcacttgtggctattgggactggaggcacccttagccacttcattcctctgatgggccc

SEQ ID NO: 27 - DNA, 457 bp, Homo sapiens
  misc_feature (217)..(217): wherein “n” is c or t
actgtcagagctgaccccagctaaggcatggcaccgcttctgtcctttctaggacctcggggtccctctgggcccagtttccctatctgtaaattggggacagtaaatgtatggggtcgcagggtgttgagtgacaggaggctgcttagccacatgggaggtgctcagtaaaggagagcaattcttacaggtgtctgcctcctgacccttccatccntcaggtgtcctgttgccccctcctcccactgacaccctccggaggcccccatgttgacagaccctcttctcctaccttgtttcccagcctgactctccttccgttctgggtccccctcctctggtcggctcccctgtgtctcatcccccggattaagccttctccgcctggtcctctttctctggtgacccacaccgcccgcaaagccacagcgcatctggatcacccgctttggtggcg

SEQ ID NO: 28 - DNA, 433 bp, Homo Sapiens
  misc_feature (193)..(193): wherein “n” is c or t
agcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgcngctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccgg

(19) United States
(12) Patent Application Publication
(10) Pub. No.: US 2012/0094842 A1
(43) Pub. Date: Apr. 19, 2012

(54) METHODS OF ASSESSING AND TREATING PULMONARY DISEASE

(76) Inventors: Peter Durie, Toronto (CA); Julian Zielenski, Gilford (CA); Mary Corey, Toronto (CA); Andrew Sanford, Vancouver (CA); Peter Pare, Vancouver (CA); Ruslan Dorfman, Thornhill (CA); Yves Berthiaume, Montreal (CA); Lap-Chee Tsui, Hong Kong (HK)

(21) Appl. No.: 12/263,510

(22) Filed: Nov. 3, 2008

Related U.S. Application Data
(60) Provisional application No. 60/984,545, filed on Nov. 1, 2007.

(51) Int. Cl. C12Q 1/68; C40B 20/00

(52) U.S. Cl. 506/2; 435/6.11

(57) ABSTRACT

Methods of diagnosing risk of pulmonary disease in a mammal with cystic fibrosis are provided comprising screening a biological sample obtained from the mammal for MBL2 or SP-A1 deficiency, and TGFB1 over-expression.

### CROSS-REFERENCE TO RELATED APPLICATIONS

This application claims the benefit of U.S. provisional application Ser. No. 60/984,545 filed Nov. 1, 2007. The entirety of which is incorporated by reference herein.

### FIELD OF THE INVENTION

The present invention relates to methods of assessing pulmonary disease in mammals having cystic fibrosis which includes the identification of mutant MBL2 or SP-A1 genes, and TGFB1 genes. The invention further relates to methods of treating mammals at risk of developing severe pulmonary disease.

### BACKGROUND OF THE INVENTION

Cystic fibrosis (CF) is an autosomal, recessive disease typically manifesting with progressive obstructive lung disease, insufficiency of the exocrine pancreas and elevated electrolyte concentration in sweat glands (OMIM #219700). Although CF is a monogenic disease, caused by mutations in the Cystic Fibrosis Transmembrane Conductance Regulator gene (CFTR), genotype-phenotype correlation studies of some manifestations suggest more complex inheritance, involving other genetic and environmental factors. In particular, the severity of pulmonary disease, which is the major cause of morbidity and mortality in CF, is poorly correlated with CFTR genotype. Patients carrying the same CFTR mutations show extremely variable lung phenotypes at all ages. It is postulated that the severity and progression of pulmonary disease in CF is modulated by secondary genetic factors called CF modifiers.

The hallmark of CF pulmonary disease is chronic infection with characteristic pathogens such as Pseudomonas aeruginosa, Staphylococcus aureus and Burkholderia cepacia, which leads to chronic inflammatory damage to lung tissue and progressive loss of pulmonary function. Genetic factors that influence susceptibility to infection are therefore of major interest. One of the first genes implicated as a pulmonary modifier in CF was the mannose-binding lectin 2 gene (MBL2). The MBL2 gene produces a protein of the collectin family, a key factor in the innate immune response. MBL2 selectively binds D-mannose and N-acetyl-D-glucosamine (GlcNAc) on bacterial surfaces and triggers MBL2-associated serine proteases (MASPs), activation of the complement pathway and eventual phagocytosis of opsonized bacteria. MBL2 also regulates the secretion of cytokines: intermediate levels of MBL2 (1-5 μg/ml) trigger secretion of pro-inflammatory cytokines (IL6, TNFα, MCP1), while high levels of MBL2 (>6 μg/ml) are known to repress secretion of pro-inflammatory cytokines and up-regulate the anti-inflammatory cytokine, IL10. On the one hand, high levels of cytokines are beneficial since they initiate pathways necessary for clearing bacterial infection, but on the other hand they can stimulate inflammatory cascades which are potentially detrimental to the lung tissue of CF patients.

MBL2 protein is known to bind to its substrates exclusively in an oligomerized form, and the collagen-like repeats are essential for the multimerization process. In the general population, levels of oligomerized MBL2 in blood vary dramatically, which is partially explained by genetic variation in the MBL2 gene. The rare X allele at position −221 of the promoter results in decreased promoter activity, while the missense mutations in exon 1 at positions 52, 54 and 57 in the collagen-like domain abolish assembly of the protein into its biologically active form. It is well documented that low levels of MBL2 in the blood are associated with susceptibility to infections in various clinical settings. Independent studies found a two-fold increase in childhood infections associated with MBL2 deficiency.

In some studies of patients with CF, MBL2 deficiency has been associated with more severe pulmonary disease and poor survival, while others have reported no association. These studies were based on relatively small CF patient cohorts (n˜100-300). A recent study of a large cohort of CF patients homozygous for the ΔF508 mutation, found no association between polymorphisms in the MBL2 gene and pulmonary function. The Drumm study was specifically designed to include age-specific extremes of pulmonary function and patients under eight years of age were excluded because of inability to define the severity of pulmonary disease. This precluded the ability to test for potential effects of the MBL2 polymorphisms at an early stage of CF lung disease.

Polymorphisms in Surfactant Protein A1 and A2 (SP-A1, SP-A2), belonging to the collectin class of proteins, play a role in respiratory distress syndrome, allergic bronchopulmonary aspergillosis and idiopathic pulmonary fibrosis. The levels of SP-A are decreased in the lungs of patients with cystic fibrosis, respiratory distress syndrome and other chronic lung diseases. These proteins are encoded by SFTPA1 and SFTPA2 genes, respectively, which are both found on chromosome 10. SP-D is another collectin present in the lung. As with mannan binding lectins, SP proteins function as multimeric complexes: SP-A1 forms 18-mers, while SP-D forms a 10-mer complex. Knockout mice for either SP-A1 or SP-D exhibit slower clearance of bacteria from lungs and, thus, defects in either gene may predispose to infections.

TGFB1 is a growth factor which plays a critical role in inflammatory processes likely to have pleiotropic effects in the lung; it prevents spontaneous activation of alveolar macrophages and secretion of pro-inflammatory cytokines; TGFB1 also causes inflammation, apoptosis, remodeling and fibrosis of lung tissue, but does not affect the efficiency of phagocytosis. Several TGFB1 gene variants were previously shown to affect the level of secreted protein and were reported to affect severity of lung disease in CF patients. Excessive tissue fibrosis in CF lungs was proposed as the basis underlying the association of the high-TGFB1-producing allele C in codon 10 of the gene with severe pulmonary CF disease.

Given the foregoing findings, it would be desirable to determine genetic variants which are predictive of pulmonary disease for patients with cystic fibrosis to permit the development of prognostic techniques and methods of treatment.

### SUMMARY OF THE INVENTION

It has now been found that over-expression of TGFB1 amplifies the effects of MBL2 and SP-A1 deficient gene mutants in mammals with cystic fibrosis. Thus, detection of the presence of TGFB1 over-expression and MBL2 or SP-A1 deficiency in mammals diagnosed with cystic fibrosis is useful for identification of mammals at risk of developing more severe pulmonary disease.

Accordingly, in one aspect of the present invention, there is provided a method of assessing pulmonary disease in a mammal with cystic fibrosis comprising:
screening a biological sample obtained from the mammal for at least one of an MBL2 or SP-A1 deficient gene mutant, and an over-expressing TGFB1 gene, wherein identification of both of said genes is indicative of a risk of severe pulmonary disease.

In another aspect of the present invention, a method of assessing pulmonary disease in a mammal having cystic fibrosis is provided comprising: determining in a biological sample obtained from the mammal expression levels of at least one of MBL2 or SP-A1, and TGFB1, in the sample, wherein a determination of MBL2 or SP-A1 deficiency and TGFB1 over-expression in comparison to MBL2, SP-A1 and TGFB1 levels in a healthy mammal is indicative of a risk of severe pulmonary disease.

In another aspect of the present invention, a method of treating a mammal at risk of developing severe pulmonary disease is provided comprising at least partially inhibiting TGFB1 in the mammal.

These and other aspects of the invention will become apparent from the detailed description that follows and by reference to the following figures in which:

### BRIEF DESCRIPTION OF THE DRAWINGS

FIG. 1 graphically illustrates normalized MBL2 levels corresponding to six MBL2 gene diplotypes: **00, XYA0, YYA0, XXAA, XYAA and YYAA in CF patients and parents;

FIG. 2 provides the Kaplan Meier curves for three MBL2 diplotypes, low MBL2-, intermediate MBL2- and high MBL2-expressing, and indicates the median age of Pseudomonas aeruginosa infection in each group;

FIG. 3 provides the Kaplan Meier curves for the three MBL2 diplotypes of FIG. 2 when combined with TGFB1 genotypes, TT (A), CT (B) and CC (C);

FIG. 4 provides the Kaplan Meier curves for non-MBL2 deficient CF patients that are carriers of the SP-A1 R219W mutation;

FIG. 5 provides the Kaplan Meier curves for non-MBL2 deficient CF patients that are carriers of the SP-A1 R219W mutation combined with different TGFB1 genotypes TGFB1-CC (A), CT (B), TT (C);

FIG. 6 provides the Kaplan Meier curves for MBL2-deficient CF patients that are carriers of SP-A1 R219W mutation (A) and Kaplan Meier curves for the carriers of the SP-A1 R219W mutation as compared with different MBL diplotypes (B);

FIG. 7 is a schematic representation of the genomic organization of the MBL2 gene (A) illustrating the position of X/Y variants at −221 nt in the promoter of the gene and structural mutations A/B, A/C and A/D at positions +161 nt, +170 nt and +154 nt in exon 1 of the MBL2 gene, and the MBL2 nucleotide sequence spanning the region encompassing the X/Y, A/B, A/C/and A/D mutations (B);

FIG. 8 is a schematic representation of the genomic organization of the SP-A1 gene (A) illustrating the position of the tested missense mutation R219W (rs4253527), and the nucleotide sequence adjacent to the mutation (B); and

FIG. 9 is a schematic representation of the genomic organization of the TGFB1 gene (A) illustrating the position of known sequence variants, including C-509T and L10P, and the nucleotide sequence adjacent to the C-509T and L10P variants is shown (B).

### DETAILED DESCRIPTION OF THE INVENTION

A method of assessing pulmonary disease in a mammal having cystic fibrosis is provided comprising screening a suitable biological sample from the mammal for at least one of an MBL2 or SP-A1 deficient gene mutant, and an over-expressing TGFB1 mutant gene. Together, the MBL2/SP-A1 and the TGFB1 mutant genes are indicative of a risk of severe pulmonary disease. The sample may alternatively be screened for MBL2 or SP-A1 deficiency, and TGFB1 over-expression, in comparison to levels found in a healthy mammal, to determine risk of severe pulmonary disease.

The term “severe” as it is used herein with respect to pulmonary disease is meant to refer to early onset or acquisition of bacterial infection as well as more rapid decline in lung function in comparison with control and diseased populations. Generally, early onset of bacterial infection or more rapid decline in lung function may be defined by values which are less than about two standard deviations below a statistical mean calculated for a given patient population.

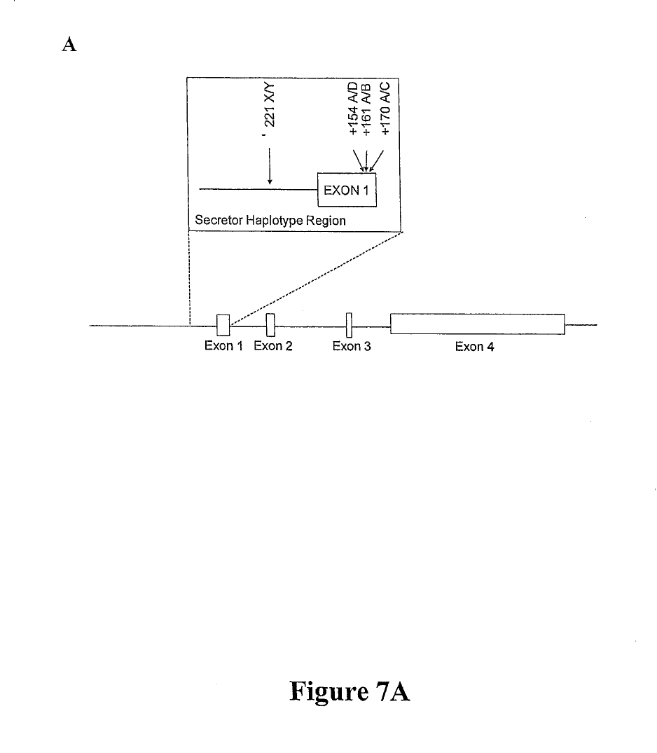

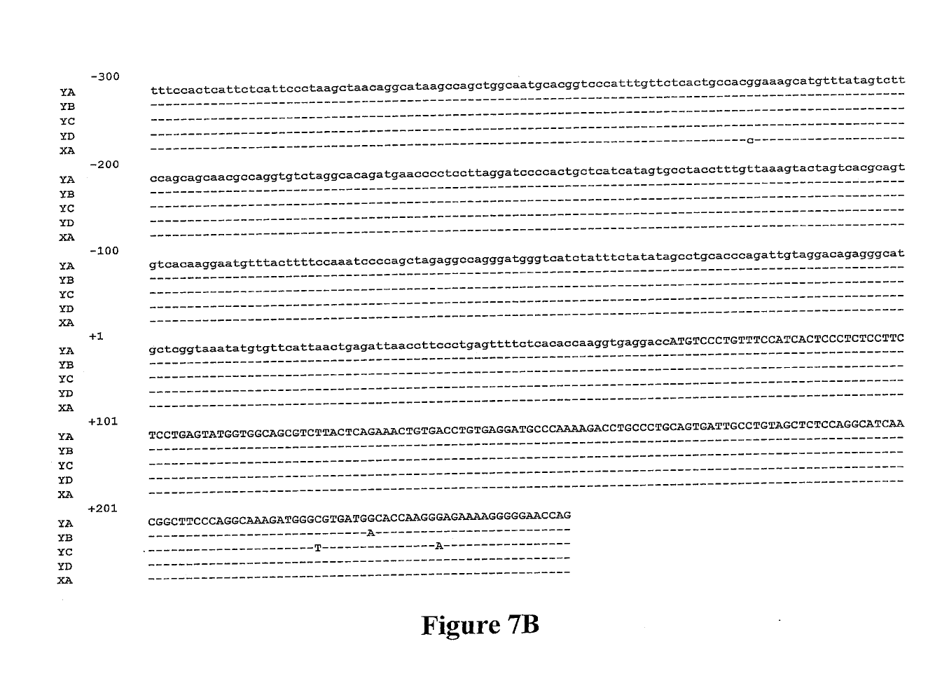

The term “deficient MBL2 mutant gene” refers to MBL2 gene alleles which include mutations that result in deficient expression of MBL2 in comparison with MBL2 expression in healthy mammals. Deficient expression of MBL2 may be as a result of an under-expressing MBL2 gene mutant or a gene mutant which expresses a dysfunctional MBL2 protein product, such as a structurally defective mutant. Generally, under-expression of MBL2 is considered to occur when MBL2 levels are reduced in comparison to MBL2 levels in healthy mammals (e.g. humans), for example, MBL2 levels which are less than about 500 μg/L. Examples of under-expressing MBL2 mutant genes include, but are not limited to, those exemplified in FIG. 7, for example, which include a mutation at at least one of positions 154, 161 and 170 of exon 1 or a mutation within the promoter at position −221, or a combination of two or more of these mutations.

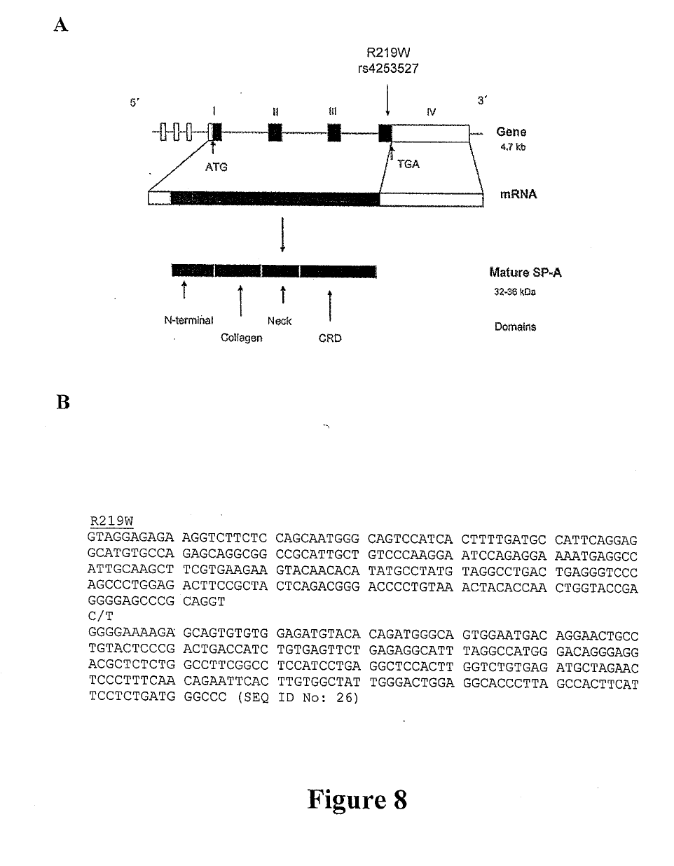

The term “deficient SP-A1 mutant gene” refers to SP-A1 gene alleles which include mutations that result in deficient expression of SP-A1 in comparison with SP-A1 expression in a healthy mammal, e.g. humans. Deficient expression of SP-A1 may be as a result of an under-expressing SP-A1 gene mutant, e.g. under-expression in an amount of less than about 500 mg/l, or due to a gene mutant which expresses a dysfunctional SP-A1 protein product, such as a structurally defective mutant. An example of a deficient SP-A1 mutant gene is one which expresses a dysfunctional SP-A1 protein such as that exemplified in FIG. 8, e.g. the R219W mutant. Other deficient SP-A1 mutant genes include, genes having a missense mutation which disrupts a Gly-X-Y repeat of SP-A1 interrupting the formation of functional SP-A1.

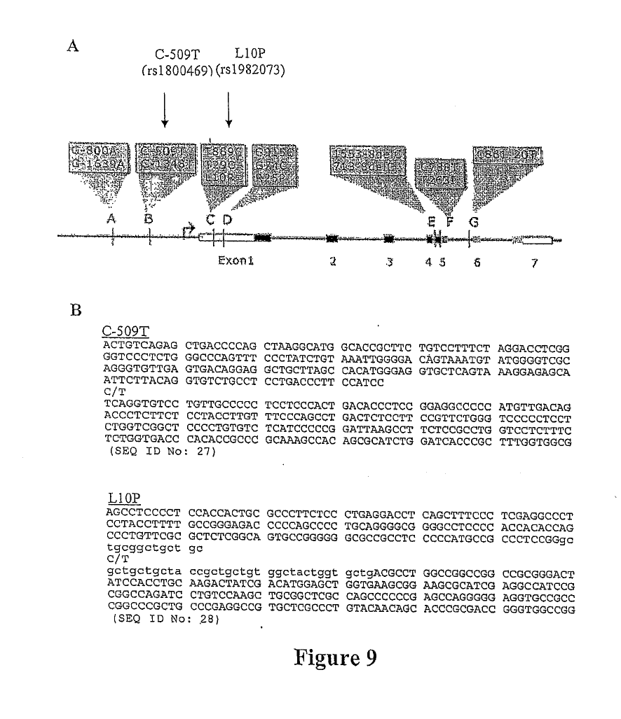

The term “over-expressing TGFB1 mutant gene” refers to TGFB1 gene alleles including mutations that result in expression of increased blood levels of TGFB1 in comparison with TGFB1 blood expression levels in a healthy mammal. Generally, levels of TGFB1 considered to be increased in comparison with TGFB1 levels in healthy mammals, e.g. humans, are levels which are greater than about 5 μg/L. Examples of over-expressing TGFB1 mutant genes include, but are not limited to, those exemplified in FIG. 9.

The term “mammal” is used herein to include both human and non-human mammals.

To conduct the method of the present invention, a biological sample is obtained that is suitable either to identify the presence of mutant MBL2 or SP-A1, and TGFB1 genes, or that is suitable to quantify the level of expression or activity of MBL2 or SP-A1, and TGFB1 protein. Suitable biological samples for the identification of genes include, but are not limited to blood, saliva, urine, semen, hair, skin, tissue biopsies, bone marrow and cerebrospinal fluid. Suitable biological samples for protein identification include blood. The sample is obtained from the mammal using methods conventional for the specific sample type. The amount of biological sample required must be sufficient to allow identification of the MBL2, SP-A1 and TGFB1 mutant genes, for example, a minimum amount of about 50 ng of genomic DNA, or an amount sufficient to allow identification of MBL2, SP-A1 and TGFB1 protein, for example, an amount of about 5 μg protein.

Prior to analyzing the biological sample, it may be necessary to process the sample to yield a form acceptable for analysis. For example, prior to analyzing the sample for the presence of MBL2, SP-A1 and TGFB1 gene mutant variants, the nucleic acid (e.g. genomic DNA) is extracted therefrom using techniques well-established in the art including chemical extraction techniques utilizing phenol-chloroform (Sambrook et al., 1989), guanidine-containing solutions, or CTAB-containing buffers. As well, as a matter of convenience, commercial DNA extraction kits are also widely available from laboratory reagent supply companies, including for example, the QIAamp DNA Blood Minikit available from QIAGEN (Chatsworth, Calif.), or the Extract-N-Amp blood kit available from Sigma (St. Louis, Mo.). In the case of a protein analysis, the sample may require purification using any one of a number of protein purification techniques, such as for example, ion exchange or size exclusion chromatography.

Once an appropriate sample is obtained, genotyping of MBL2, SP-A1 and TGFB1 genes may be conducted to identify mutant MBL2, SP-A1 and TGFB1 genes using a variety of methods including, but not limited to, multiplexed microarray bead-based technology and TaqMan genotyping assays.

Alternatively, MBL2, SP-A1 and TGFB1 protein or activity levels within the sample are determined. Protein levels are measured by assaying for protein expression by measuring protein concentration. As one of skill in the art will appreciate, various techniques may be employed to measure protein concentration. For example, for quantification of active MBL2 and SP-A1 levels, detection of the active oligomerized form of the protein may be determined using microarray bead-based technology. For quantification of TGFB1, an ELISA test may be used.

The method of the present invention, thus, provides a means for the early detection of patients at risk of developing “severe” pulmonary disease. Genetic testing for MBL2, SP-A1 and TGFB1 gene variants may permit such early identification of “at risk” patients either at diagnosis or through newborn screening. Alternatively, detection of MBL2, SP-A1 and TGFB1 protein levels may allow identification of “at risk” patients. This would allow physicians to closely monitor an “at risk” patient and to treat the patient accordingly, for example, employing more aggressive efforts to prevent early onset of bacterial lung infection.

In another aspect of the present invention, a method of treating a mammal with pulmonary lung disease, such as lung tissue fibrosis associated with bacterial infection. The treatment method comprises inhibiting over-expression of TGFB1 in a mammal to be treated. As one of skill in the art will appreciate, expression of TGFB1 may be inhibited at the nucleic acid level as well as at the protein level. In either case, the result of inhibiting, or at least reducing, TGFB1 expression is achieved. The term “inhibit” as it used herein with respect to TGFB1 is meant to refer to any reduction of TGFB1 activity, including both complete as well as partial inhibition, by which the amplification effect on MBL2 or SP-A1 under-expression is prevented or at least reduced.

TGFB1 gene expression may be inhibited at the nucleic acid level using well-established methodologies such as anti-sense and siRNA technologies. Thus, antisense oligonucleotides based on the TGFB1 gene sequence may be designed which are effective to bind to TGFB1-encoding nucleic and inhibit the expression thereof. The term “antisense oligonucleotide” as used herein means a nucleotide sequence that is complementary to at least a portion of a target TGFB1 nucleic acid sequence. The term “oligonucleotide” refers to an oligomer or polymer of nucleotide or nucleoside monomers consisting of naturally occurring bases, sugars, and intersugar (backbone) linkages. The term also includes modified oligonucleotide structures such as oligonucleotides including a modified phosphorous backbone, or substituted oligomers comprising non-naturally occurring monomers or portions thereof, and oligonucleotide analogues such as peptide nucleic acids (PNA), which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells) as well as the antisense binding region. In addition, two or more antisense oligonucleotides may be linked to form a chimeric oligonucleotide.

Small molecules of RNA (siRNA) may also be used which function to disrupt translation of TGFB1 and thereby reduce expression of TGFB1. Application of siRNA fragments that correspond with regions in the TGFB1 gene are effective to block TGFB1 expression by binding to the TGFB1 gene and thereby prevent translation of the gene to yield functional TGFB1. As one of skill in the art will appreciate, useful siRNA fragments need not correspond exactly with a TGFB1 target gene, but may incorporate sequence modifications, for example, addition, deletion or substitution of one or more of the nucleotide bases therein, provided that the modified siRNA retains it ability to bind to the target TGFB1 gene. Selected siRNA fragments may additionally be modified in order to yield fragments that are more desirable for use. For example, siRNA fragments may be modified to attain increased stability in a manner similar to that described for antisense oligonucleotides.

In addition, double stranded oligodeoxynucleotides may be employed which mimic transcription factor binding sites. These attract and bind TGFB1 transcription factors, reducing the binding of transcription factor to turn on transcription of the mutant TGFB1 gene in the mammal, and thus lowering expression of TGFB1.

Oligonucleotides useful in the present treatment protocol may be made using chemical synthesis and enzymatic ligation reactions well-established in the art and generally implemented using automated systems. Such oligonucleotides may also be produced biologically. In this regard, a coding nucleic acid sequence is incorporated into an expression vector that is then introduced into cells in the form of a recombinant plasmid, phagemid or attenuated virus in which antisense/RNA sequences are produced under the control of a high efficiency regulatory region. The desired oligonucleotides may then be harvested for use.

Once prepared, the oligonucleotides may be introduced into tissues or cells using known techniques such as microinjection and electroporation. Delivery systems include, but are not limited to liposomes and other polyplexes, viral vectors such adenoviral and retroviral vectors and dendrimers.

Inhibition of TGFB1 expression may also be accomplished using TGFB1-specific immunological inhibitors such as monoclonal antibodies prepared using well-established hybridoma technology such as that developed by Kohler and Milstein (Nature 256, 495-497(1975)). Hybridoma cells can be screened immunochemically for production of antibodies specifically reactive with TGFB1 and these antibodies can then be isolated. The term “antibody” as used herein is intended to include both antibody molecules and fragments thereof which specifically react with the TGFB1 protein to inhibits its expression according to the invention, as well as chimeric antibody derivatives, i.e. antibody molecules resulting from the combination of a variable non-human animal peptide region and a constant human peptide region. It will also be appreciated that antibodies in accordance with the present invention may be modified to increase the biological stability thereof or to increase the physical affinity thereof for TGFB1. For example, antibodies may be combined with PEG or other synthetic agents to enhance affinity or stability.

Alternatively, inhibition of TGFB1 expression may be accomplished using a soluble form of a TGFB1 receptor complex such as TGFR Type II or any other form of scavenger molecule specific to TGFB1 which is capable of binding the TGFB1 protein to minimize or prevent its activity.

Once prepared, antibodies and other biological inhibitory molecules and complexes may be introduced by intravenous infusions combined with appropriate carriers or adjuvants as described in more detail herein.

Inhibition of TGFB1 function may also be accomplished by administration of synthetic small molecule inhibitors including peptide mimetics, for example, based on known biological inhibitors, but which incorporate desirable features such as protease resistance. Generally, such inhibitors are designed based on techniques well-established in the art, including computer modeling. TBGB1 inhibitors may be designed to inhibit the TGFB1 signaling pathway or TGFB1 receptor binding, for example, to the TGFB1 receptor type II. Alternatively, kinase inhibitors of SMAD2, 3 or 4 phosphorylation may be used or designed for use to prevent phosphorylation of TGFB1, which is required for activity, or protease inhibitors that block TGFB1 processing and activation.

TGFB1 inhibitors may be administered in combination with a pharmaceutically acceptable carrier. The expression “pharmaceutically acceptable” means acceptable for use in the pharmaceutical and veterinary arts, i.e. not being unacceptably toxic or otherwise unsuitable. Examples of pharmaceutically acceptable carriers include diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Aerosol formulations, for example, for nasal delivery, may also be prepared in which suitable propellant adjuvants are used. Other adjuvants may also be added to the composition regardless of how it is to be administered, for example, anti-microbial agents may be added to the composition to prevent microbial growth over prolonged storage periods.

The treatment protocol may optionally include the administration of a therapeutically effective amount of MBL2 or SP-A1, for example, an amount sufficient to bring the plasma level of MBL2 or SP-A1 in a mammal being treated to at least the normal level found in healthy mammals (e.g. above about 500 μg/L), and preferably to a level that may be effective to prevent the onset of pulmonary disease. The MBL2 or SP-A1 may be recombinant, prepared using well-established techniques in the art, or may be obtained by purification from blood.

The treatment protocol may also optionally include the administration of other therapeutic agents, for example, which are effective to treat lung disease, including antibiotics such as TOBY, azythromycin, or other mucus clearing treatments such as Dornase Alfa, Denufosol and hypertonic saline in nebulized form

Embodiments of the invention are described by reference to the following specific examples which are not to be construed as limiting.

### EXAMPLE 1

### Material and Methods

### Cystic Fibrosis Patients and Parent Cohorts.

Cystic fibrosis patients and their family members were recruited from 37 specialized CF clinics across Canada. They participate in the Canadian CF Modifier Study. The protocols for this study were approved by ethical review boards at the Hospital for Sick Children and all participating institutions. Informed consent was obtained from each individual or his/her guardian. The Canadian CF Modifier Study population consists of 2441 patients diagnosed with CF between 1951 and 2006 and 3092 parents. This report of first infection as a phenotype was restricted to pediatric patients (less than 18.5 years of age), in order to capture the most reliable information on first infection, and to compute average lung function decline unbiased by mortality selection. The age of first infection was generally not available in the records of patients reported from clinics specializing in adult CF care. CF patients included in this study had pancreatic insufficiency (PI) and carried severe CFTR mutations on both alleles. Patients reported as PI but carrying at least one CFTR mutation typically associated with pancreatic sufficiency (PS) were excluded. The demographic and clinical characteristics of the pediatric patient cohort are shown in Table 1.

### Pulmonary Phenotypes

To evaluate MBL2, SP-A1 and MBL2/SP-A1 (stratified by TGFB1 variants) as a pulmonary CF modifier, two lung-related, clinical outcome variables were used: age at first Pseudomonas aeruginosa infection and forced expiratory volume in one second (FEV₁). Patients attending Canadian CF clinics have sputum cultures and lung function testing every 3 months. Age of first Pseudomonas aeruginosa infection was used as an indicator of susceptibility to infection with this bacterium. FEV₁, as a percent of predicted for sex, height and age, reflects the progression of CF lung disease and is the best predictor of survival. Predicted values were based on the formula of Knudson et al. (1983. Am Rev Respir Dis 127:725-734) for those over the age of 10 years and Corey et al.(1976. Am Rev Respir Dis 114:1085-1092) for those 6 to 10 years of age. FEV₁ measurements in the 3 years prior to enrollment were recorded in order to capture the current status and rate of decline for each patient.

### Plasma and DNA Samples

Individual blood samples were collected in glass tubes containing anticoagulant (Acid Citrate Dextrose; ACD-Vacutainer tubes, BD) and maintained at room temperature. Plasma samples were isolated by centrifugation at 1500 rpm for 10 minutes and immediately frozen and stored at −70° C. Genomic DNA was extracted using the phenol/chloroform procedure (Cutting et al. 1989. Am J Hum Genet 44:307-318). The DNA stocks were stored at 4° C.

### Genotyping

### MBL2 Gene

Four functional MBL2 gene variants were genotyped in genomic DNA samples: the promoter variant at position −221 (Y or X variant) and three structural mutations in exon 1 (B, C and D variants) as shown in Table 2. The genotyping was performed using Luminex Bead Array technology implemented for the MBL2 gene variants as described in Example 2. MBL2 diplotypes (Table 2) were inferred from promoter variants (X, Y) and structural variants in exon 1 (A, B, C, D) according to Madsen et al, 1995. J Immunol 155:3013-3020. (Table 2).

### SP-A1 Gene

The genotyping was performed using Luminex Bead Array technology implemented for the SP-A1 R219W mutant.

### TGFB1 Gene

The codon 10 expression variant (rs1982073, L10P) in the TGFB1 gene was genotyped using TaqMan genotyping assay (Livak et al. 1995. Nat Genet 9:341-342). Genotyping of the promoter variant (−509; rs1800469) was performed using Luminex Bead Array technology (Luminex Corporation).

### Measurement of MBL2 Protein Plasma Levels

The plasma level of oligomerized MBL2 protein were measured in blood samples from 1393 CF patients and 600 parents as described in Example 3. The MBL2 protein levels were normalized to adjust for batch differences, using the mean and standard deviation for 10 non-CF control samples included in every batch. Therefore the normalized MBL2 expression level reflects the variation above or below that expected for samples unrelated to CF.

### Statistical Analyses

Patients who had not acquired Pseudomonas aeruginosa infection at the time of their study visit will likely acquire the infection in the future; therefore, their age at first infection is unknown. To account for this, age at first infection was treated as a right-censored variable and time-to-event analysis was used to compare age at first infection in different subgroups. Kaplan Meier survival curves were plotted to demonstrate the distribution of age at first Pseudomonas aeruginosa infection, and curves were compared using the log-rank statistic. Pseudomonas aeruginosa-free survival curves were compared for MBL2 genotype groups, SP-A1 R219W mutant, TGFB1 genotypes, and for MBL2/TGFB1 and SP-A1/TGFB1 combinations. Cox proportional hazards regression was used to test the effects of sex and of multiple genotype groups on age at first infection.

Pulmonary function testing involves volitional breathing maneuvers that are generally not possible in children until 5 to 7 years of age. Current lung function and decline over the past 3 years, represented by FEV₁ % predicted, were compiled for patients who had been tested. Longitudinal pulmonary function patterns (in patients with at least 2 measurements) were analyzed using a random effects mixed model regression, as described by Schluchter et al. (2006. Am J Respir Crit Care Med 174:780-786) and the mean intercept at 10 years of age and the mean slope were compared in potential modifier categories. The dependent variable in all models was FEV₁ % predicted and the time variable was age-10. Genotype groups for potential modifiers were included as categorical variables. Interaction terms for modifier categories and age provided a statistical test for category differences in the change of lung function over time. The effects of gender and age were also added to selected models, to address previously reported findings.

All statistical analyses were performed using SAS version 9.1 (SAS Institute Inc. 2004. User's Guide. Cary N C: SAS Institute Inc.: SAS Institute, Inc.).

### Results

### Demographic and Clinical Characteristics of the Pediatric CF Patient Cohort

The cohort of 1019 CF pediatric patients is summarized in Table 1. The largest group of CF patients sharing the same CFTR genotype consists of those homozygous for the deltaF508 mutation (Tsui, L. C. 1992. Hum Mutat 1:197-203). This severely affected CF group serves as a clinical reference for patients with other CFTR genotypes. The demographic and clinical parameters in CF patients homozygous for ΔF508 was compared with the rest of the PI group (“Other PI”) as shown in Table 1 below. None of the demographic and clinical parameters were significantly different between the two groups.

TABLE 1
Demographic and clinical characteristics of CF patients
(age < 18.5 years) with pancreatic insufficiency (PI).

| | All | By CFTR mutation(s) | | |
|---|---|---|---|---|
| | Patients | ΔF508/ΔF508 | Other PI^{A} | |
| | (n = 1019) | (n = 611) | (n = 408) | P-value^{B} |
| Age mean(yrs) | 10.5 | 10.5 | 10.5 | 0.94 |
| Median Age of | 0.36 | 0.35 | 0.38 | 0.35^{C} |
| diagnosis(yrs) | | | | |
| Meconium ileus | 21.2% | 22.3% | 19.6% | 0.35 |
| CF liver | 4.4% | 3.8% | 5.4% | 0.22 |
| disease | | | | |
| CF related | 2.8% | 2.9% | 2.7% | 0.85 |
| diabetes | | | | |
| Mean FEV1% | 84.1 | 83.7 | 84.8 | 0.50 |
| pred | (n = 801) | (n = 483) | (n = 318) | |
| P. aeruginosa | 69.0% | 67.3% | 71.6% | 0.15 |
| positive | | | | |
| Median age of | 7.2 | 7.5 | 6.9 | 0.13^{D} |
| first P.a. (yrs) | | | | |
| B. cepacia | 5.5% | 5.9% | 4.9% | 0.57 |
| positive | | | | |

^{A}Other PI group includes ΔF508/other and other/other “severe” genotypes
^{B}ΔF508/ΔF508 versus “Other PI”, Student t test or Fisher exact test except:
^{C}Wicoxon rank sum test
^{D}Log rank test of time to event

### MBL2 Gene Variants and Genotypes

Four MBL2 gene variants (Table 2) were typed in 1393 CF patients and 600 randomly selected parents from the entire CF Modifier Study cohort. The overall allelic and genotypic frequencies were similar in the patient and parent cohorts. Six major diplotypes were established from family members' genotypes at four variant sites (Table 2). Their frequencies were similar in patients and parents.

TABLE 2
MBL2 gene variants: genotypes, diplotypes and notation
MBL2 GENE VARIANTS AND NOTATION

| | | Nucleotide | Amino acid | rs |
|---|---|---|---|---|
| Variant | Location | Variant | change | number |
| Y^{A} | Promoter | −221G | — | — |
| X | Promoter | −221C | — | 7096206 |
| A^{A} | Exon 1 | 154C, 161G, 170G | Arg52, Gly54, | — |
| | | | Gly57 | |
| B^{B} | Exon 1 | 154T | Cys52 | 5030737 |
| C^{B} | Exon 1 | 161A | Asp54 | 1800450 |
| D^{B} | Exon 1 | 170A | Glu 57 | 1800451 |

MBL2 DIPLOTYPES

| | Predicted genotype | Simplified diplotype |
|---|---|---|
| Diplotype | combinations^{C} | notation |
| Diplotype 1 | Y0/Y0 + X0/Y0 | **00^{D} |
| Diplotype 2 | XA/Y0 | XYA0^{E} |
| Diplotype 3 | YA/Y0 | YYA0 |
| Diplotype 4 | XA/XA | XXAA |
| Diplotype 5 | XA/YA | XYAA |
| Diplotype 6 | YA/YA | YYAA |

MBL2 DIPLOTYPE GROUPS

| | | Median MBL2 |
|---|---|---|
| Diplotype group | Diplotypes | expression level |
| Diplotype group 1 | Y0/Y0 + X0/Y0 + XA/Y0 | low |
| Diplotype group 2 | YA/Y0 + XA/XA | intermediate |
| Diplotype group 3 | XA/YA + YA/YA | high |

^{A}wild-type;
^{B}due to the same structural effect on the MBL2 protein the B, C and D variants were combined as the 0 variant group.
^{C}The phase prediction of the B, C and D structural mutations with the XY variant in the promoter region (−221) were based on the very high LD between these mutations and Y allele of the XY variant.
^{D}the extremely rare diplotype XY00 was combined with the YY00 genotype as the **00 diplotype.
^{E}The diplotype 2 was inferred.

### MBL2 Protein Levels in MBL2 Diplotype Groups

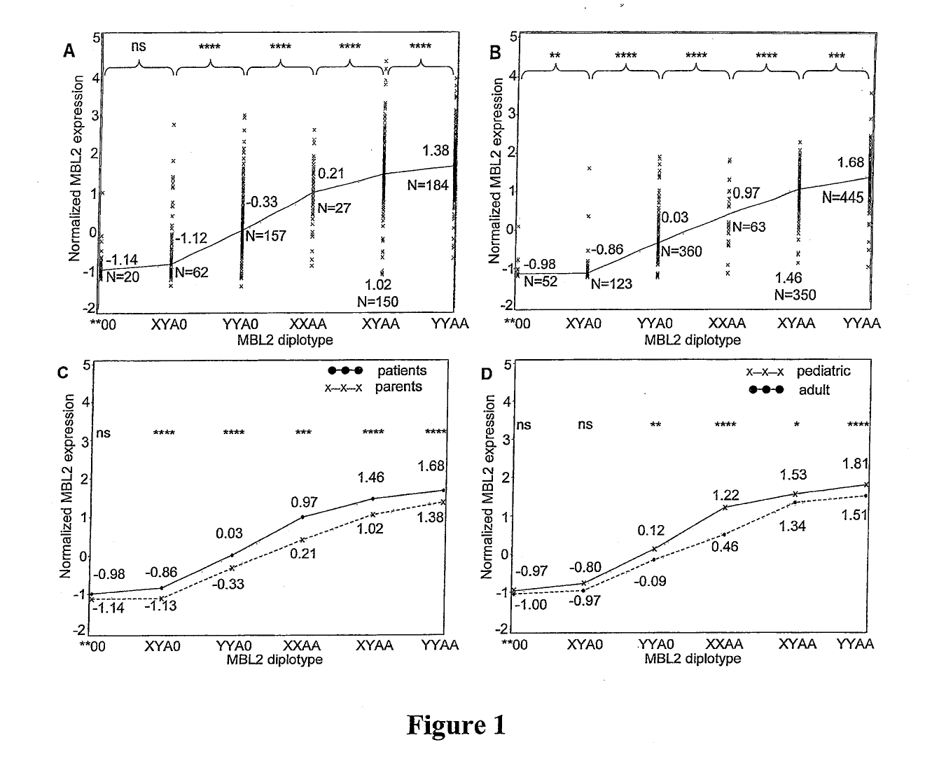

The plasma concentration of oligomerized MBL2 protein was measured in the 1393 CF patients and 600 parents for whom the diplotypes had been established. Expression of MBL2 oligomers and MBL2 genotypes were significantly correlated. The quantity of MBL2 protein in both the parent group (FIG. 1A) and CF patients (FIG. 1B) strongly correlated with the six diplotype groups (Table 2).

The normalized MBL2 expression increased with the proportion of A and Y alleles in these diplotypes, from 00_**, A0_XY, A0_YY, AA_XX, AA_XY to AA_YY diplotypes (FIG. 1). The functional consequences of structural mutations in exon 1 which prevent MBL2 assembly (combined as the 0 allele), were more pronounced than the corresponding promoter variant X that is associated with lower MBL2 expression. This observation can be explained by the fact that homozygosity for any of the structural mutations or their compound heterozygotes prevents oligomerization of MBL2 while homozygosity for the X allele reduces production of the MBL2 oligomer. However, considerable variation in the expression was observed for various diplotypes, except for the **_{—}00 diplotype (FIGS. 1A and 1B) where presence of two structural mutations (00) completely prevented oligomerization of MBL2.

The lowest level of MBL2 expression in CF and parent cohorts was associated with two inferred diplotypes: **_{—}00 and XY_A0. Pair-wise analysis demonstrated that MBL2 expression was not statistically different between individuals carrying the **_{—}00 and XY_A0 diplotypes. Therefore, the “low” MBL2 diplotype group (low-MBL2 expression and MBL2-deficient diplotype group) were combined. Similarly, at the other extreme, the two diplotypes corresponding to the highest expression, AA_XY and AA_YY were combined into the “high” MBL2 diplotype group. The remaining two diplotypes demonstrating intermediate levels of MBL2 expression including the rare XXAA diplotype, (present in 3.7% of patients) and the more common (25.8%) diplotype YYA0, were merged into an “intermediate” MBL2 diplotype group. The range of expression of the XXAA diplotype was similar to the range of the YYA0 diplotype, despite a significant difference in the median values (p<0.0001).

### MBL2 Expression in CF Patients and Parents

Non-parametric analysis of the median normalized MBL2 expression between CF patients and the parent (control) group demonstrated that CF patients have significantly higher levels of MBL2 protein (FIG. 1C). The higher MBL2 expression in CF patients is most likely associated with induction of MBL2 due to the persistent bacterial challenge and mobilization of innate defense mechanisms. The lack of a significant increase in MBL2 expression in the 00_** diplotypes almost certainly results from the structural mutations that prevent oligomerization of the protein which in turn leads to an inability to detect the MBL2 oligomer in the assay system.

### MBL2 Expression in the Pediatric and Adult Cohorts

To investigate a potential effect of age on MBL2 expression, the CF patients were divided into pediatric (<18.5 years of age; n=848) and adult (=>18.5 years of age; n=545) groups. The pediatric group had significantly higher plasma levels of MBL2 than the adult group for all diplotypes except 00_** (FIG. 1D). Higher expression of MBL2 in pediatric patients could be attributed to induction of the MBL2 complement pathway as an early (innate) defense against opportunistic bacteria infecting younger patients.

MBL2 and Age of First Infection with Pseudomonas Aeruginosa

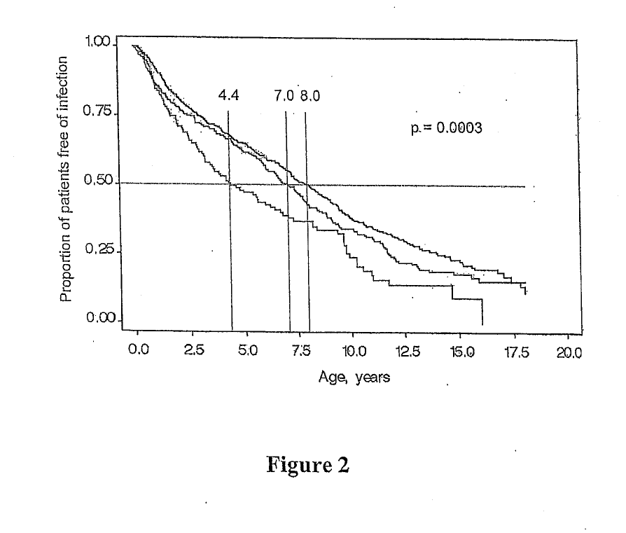

FIG. 2 shows the Kaplan Meier curves for the three MBL2 diplotypes in the pediatric cohort. The median age of first Pseudomonas aeruginosa infection was significantly different for low, intermediate and high expressing MBL2 groups (4.4, 7.0 and 8.0 years respectively, p=0.0003), indicating that MBL2 deficiency is associated with significantly earlier onset of Pseudomonas aeruginosa infection.

Similar analysis on a sub-cohort that excluded all MBL-deficient individuals indicated that the R219W polymorphism in the SP-A1 gene also significantly increased the age of first infection with Pseudomonas aeruginosa. For the carriers of the R219W allele the median age of infection was 6.4 years while median for non-carriers was 7.8 years (p=0.029) The effect of SP-A1 mutation was independent of MBL deficiency as in MBL patients there was no additive effect of SP-A1 mutation.

### Polymorphisms in the TGFB1 Gene Influences the Age of Onset of First Pseudomonas Aeruginosa Infection in the MBL2-Deficient CF Patients

High expressing variants of the transforming growth factor beta 1 (TGFB1) gene have been shown to be associated with a more severe lung disease phenotype. To investigate if this could be explained by early Pseudomonas aeruginosa infection, age of first infection for the TGFB1 codon 10 (rs1982073) genotypes was analyzed in the pediatric CF cohort. There was no difference in the age of first Pseudomonas aeruginosa infection between the genotypes (P=0.7).

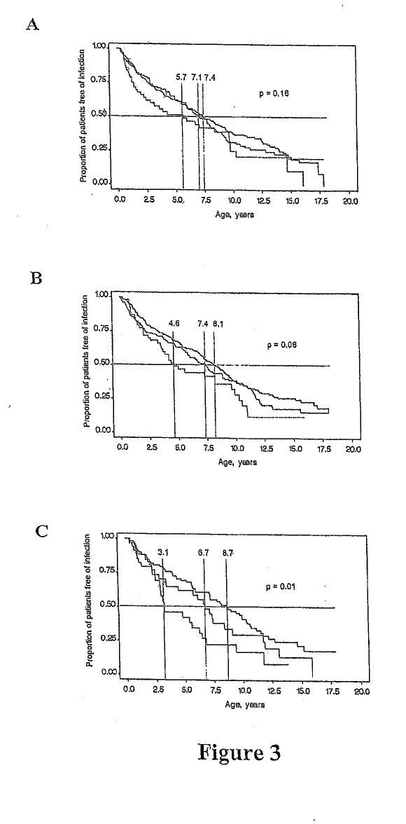

Analysis of the MBL2 diplotype groups stratified by the TGFB1 codon 10 genotypes demonstrated an influence of the high-expressing TGFB1 allele C on age of first Pseudomonas aeruginosa infection among the three MBL2 diplotype groups. In this regard, no effect of MBL2 diplotype on age of first infection was observed in the subset of patients carrying the low TGFB1-expressing genotype TT (FIG. 3A; P=0.16). However, in the subjects homozygous for the high-expressing TGFB1 C allele the differences in median age of first Pseudomonas aeruginosa infection by MBL2 diplotype group were magnified (3.1, 6.7 and 8.7 years in low, med and high expressing groups respectively, p=0.01, FIG. 3C). In those who had low levels of MBL2, the high-expressing TGFB1 genotype (CC) was associated with the earliest infection age while the low-expressing TGFB1 genotype (TT) was associated with the oldest age at first infection. The TGFB1 gene variant (rs2241715, incorporated herein by reference) exhibited a similar result as those observed for the codon 10 variant (data not shown). Although this observation suggested the possibility of gene-gene interaction between MBL2 and TGFB1 genes, testing by the Cox proportional hazards regression model failed to detect such interaction (P=0.2 for the interaction term MBL2* TGFB1).

Polymorphisms in the TGFB1 Gene Influences the Age of Onset of First Pseudomonas Aeruginosa (P.a.) Infection in the CF Patients with SP-A1-R219W Mutant Allele.

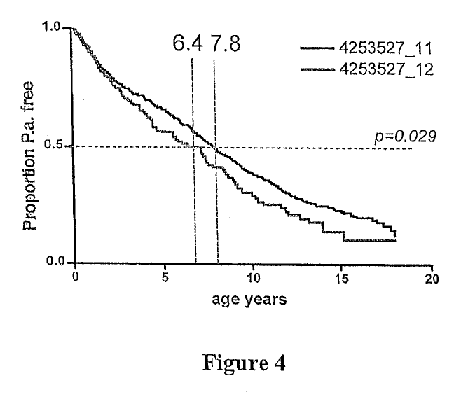

FIG. 4 shows the Kaplan Meier curve for the two SP-A1 genotypes (wild-type and heterozygous R219W mutant) in the pediatric cohort. The median age of first Pseudomonas aeruginosa infection was significantly different for the R219W mutant (6.4 years) than for the wild-type (7.8 years), indicating that SP-A1 deficiency is associated with significantly earlier onset of Pseudomonas aeruginosa infection.

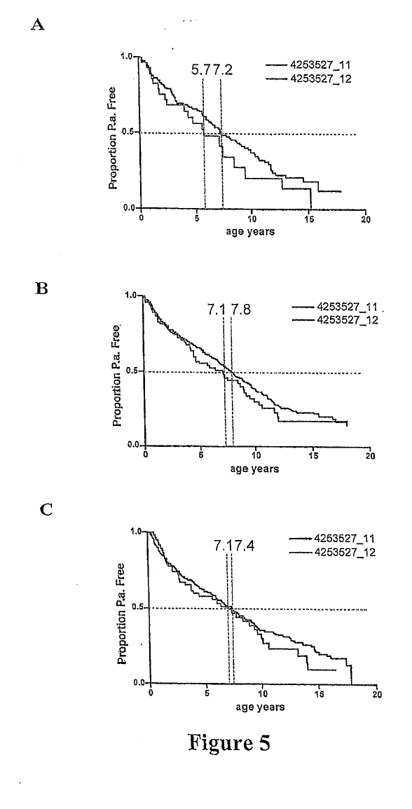

Similar to MBL2 deficiency, the reduction in SP-A1 function was modulated by the TGFB1 genotype, where a combination of the SP-A1 mutation and TGFB1 genotype CC greatly increased the risk of early infection. In patients with the TGFB1 CC genotype that were carriers of the R219W allele, the median age of first P.a. infection was 5.7 years versus 7.2 for patients with wild-type SP-A1 genotype (FIG. 5A). In CF patients with the TGFB1 low-producing genotype (TT), the SP-A1 mutation had no apparent effect (FIG. 5C). Therefore, the risk of infection is greater in patients with the SP-A1 R219W allele if they have high-producing TGFB1 genotype.

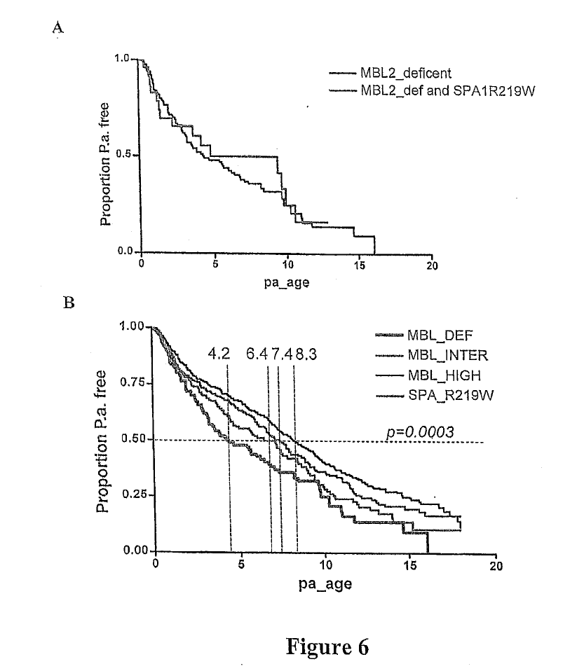

The median age of first Pseudomonas aeruginosa infection was compared between patients having MBL2 deficient mutations, and patients having MBL2 deficient mutations and the SP-A1 R219W mutation (FIG. 6A). FIG. 6B illustrates effect of the SP-A1 R219W mutation on median age of first Pseudomonas aeruginosa infection in comparison with 3 MBL diplotypes groups as set out in Table 2.

### MBL2 and MBL2/TGFB1 Modulation of Pulmonary Function in CF Patients

Five hundred and eleven patients were old enough to have multiple pulmonary function measurements in the 3 years before study enrollment. In a mixed regression model of FEV₁ % predicted vs. age, MBL2, TGFB1, and all interaction terms, the 3-way interaction was significant (p=0.002) indicating that the slope of FEV₁ vs. age was affected by MBL2 and TGFB1 genotypes. Table 3 shows how the interaction of these two modifiers affects lung function.

TABLE 3
Mixed model analyses of FEV₁ % predicted vs. age and MBL2 diplotype group in
pediatric CF patients (<18.5 years).

| | | | | Codon 10 = TT | | | Codon 10 = CT | | | | | |
|---|---|---|---|---|---|---|---|---|---|---|---|---|
| | All patients^{A} | | | (197) | | | (233) | | | Codon 10 = CC (81) | | |
| | (n = 511) | | | (n = 197) | | | (n = 233) | | | (n = 81) | | |
| MBL2 group | Age 10^{B} | Slope^{C} | n | Age 10 | Slope | n | Age 10 | Slope | n | Age 10 | Slope | n |
| Low MBL2 | 85.1 | −2.57 | 62 | 83.9 | −2.32 | 30 | 88.4 | −1.00 | 21 | 81.3 | −5.42 | 11 |
| Med MBL2 | 87.4 | −1.82 | 160 | 86.7 | −1.26 | 61 | 85.8 | −1.53 | 78 | 95.1 | −3.46 | 21 |
| High MBL2 | 87.2 | −1.56 | 289 | 86.2 | −1.99 | 106 | 88.5 | −1.09 | 134 | 86.4 | −1.55 | 49 |
| p-value | 0.70 | 0.05 | | 0.80 | 0.36 | | 0.65 | 0.56 | | 0.11 | 0.0002 | |

^{A}Patients with at least 2 measurements of FEV₁ between 6 and 18 years of age.
^{B}Mixed model estimate of mean FEV₁ % predicted at age 10 years.
^{C}Mixed model estimate of mean slope of FEV₁ % predicted vs. age.
In a model based on age, TGFB1 codon 10 genotype, and MBL2 genotype group, there was a 3-way interaction (p = 0.002) of slope with MBL2 and TGFB1. Therefore separate models of FEV1 vs. age and MBL2 were computed for each level of TGF codon 10

Rate of decline of FEV₁ was significantly steeper in the MBL2 diplotype groups associated with low expression (p=0.05), consistent with the earlier acquisition of Pseudomonas aeruginosa. When stratified by TGFB1 genotype, the significant effect of MBL2 on FEV1 was only seen in the high expressing CC genotype group (p=0.0002). The intercept at age 10 was relatively stable across all genotype combinations, except in the high TGFB1/low MBL2 group, where it was significantly lower, consistent with the earliest age at first Pseudomonas aeruginosa infection. Analysis of TGFB1 codon 10 alone showed a significant effect on lung function, consistent with earlier reports. Rate of decline in FEV1 was −2.63, −1.25, and −1.95% predicted per year in CC, CT and TT genotypes respectively, p=0.0005.

Taken together, these data suggest that the effect of MBL2 or SP-A1 deficiency is a critical determinant of the severity of lung disease at a young age in patients with CF disease and high expressing variants of TGFB1 enhance this effect.

### The Effect of Gender on Age at First Infection and Lung Function

The age at first Pseudomonas aeruginosa infection was significantly lower in females than in males (log rank p=0.04, median age at first Pseudomonas aeruginosa was 6.7 for females vs. 7.9 for males). However there was no interaction between gender and MBL2 diplotype group (p=0.62) or TGFB1 codon 10 (p=0.98) in a Cox regression model.

Consistent results were seen for lung function. Females showed a more rapid mean rate of decline in FEV1 than males (−1.45 vs. −1.08% predicted per year, p<0.0001), but there was a similar intercept at age 10 years (87.3 vs. 86.7, p=0.73). Sex and the 3-way interaction of sex*age*genotype were added to mixed models of FEV1 based on MBL2 or TGFB1, and there was no suggestion that the effects of either were different for males and females. The three-way interaction terms were not significant in the MBL2 model (p=0.80) or in the codon 10 model (p=0.41). The effect of gender on the interaction between TGFB1 codon10 and MBL2 diplotype groups for lung function decline could not be tested. This regression model involves a four-way interaction and all possible two- and three-way interactions and the numbers of patients in the smallest categories of sex*MBL2*TGFB1 were insufficient.

### EXAMPLE 2

### Genotyping

### MBL2 and SP-A1

The target SNPs were amplified in multiplexed PCR reactions using a HotStar Taq Polymerase Kit (Qiagen) with the following conditions: 2.5 mM MgCl₂, 0.25 mM dNTPs, 10 nmole of each primer and 0.25 U of the polymerase and 50 ng of genomic DNA. For some multiplexed PCR reactions 1× Q solution was added to increase the specificity of reaction (Table 4). The MJ Research PTC 225 Thermocycler conditions were as follows: initial activation at 95° C. for 15 min, followed by 32 cycles of 94° C. for 30 sec, 59° C. for 90 sec, 72° C. for 1 min. The amplicons were treated with mixture of Shrimp Alkaline Phosphatase and Exonuclease (1 U/20 μl reaction each) for 1 hour at 37° C. and 95° C. for 5 min. The Allele Specific Primer Extension (ASPE) reactions were performed using Platinum Tsp Polymerase (Invitrogen) with 1.5 mM MgCl₂, 0.25 mM dNTPs (except biotin-dCTP at 0.1 mM) and mixed ASPE primers (0.1 pmole each, Table 4). The cycler conditions were as follows: initial denaturation at 95° C. for 2 min, followed by 16 cycles of 94° C. for 10 sec, annealing at 62° C., decreasing 0.5° C. with each cycle for 1 min, extension at 72° C. for 30 sec, followed by 16 cycles of 94° C. for 10 sec, annealing at 55° C. for 1 min, extension at 72° C. for 30 sec. The ASPE reactions were hybridized with Luminex FlexMap100 beads (1250 of each bead/well) in 50 μl 1× Wash Buffer (10× contains 2M NaCl, 1M Tris, 0.8% Triton X-100). Hybridization was performed 92° C. for 2 min followed by 37° C. for 30-60 min. The hybridized beads were filtered through a Millipore MultiScreen filter plate on a vacuum station and were washed once with 150 μl of 1× Wash Buffer and developed with Streptavidin-Phycoerythrin (0.15 μl/reaction) in 150 μl of 1× Wash Buffer with shaking for 10 min. The reactions were analyzed on the Luminex LiquiChip Flowcytometry Reader. The raw data were exported to the internal database where alleles were annotated using a proprietary algorithm.

The genotyping data generated by the method described above was verified by sequencing.

TABLE 4
PCR and ASPE Primer sequences

| | |
|---|---|
| PCR primers | |
| MBL_ex1F | CCTGTAGCTCTCCAGGCATC (SEQ ID No: 1) |
| MBL_ex1R | CAGGCAGTTTCCTCTGGAAG (SEQ ID No: 2) |
| MBL_XY promoterF | CACCTGGGTTTCCACTCATT (SEQ ID No: 3) |
| MBL_XY promoter | CCTTGTGACACTGCGTGACT (SEQ ID No: 4) |
| rs1800469/TGEb1(-509)F | GTTGAGTGACAGGAGGCTGCTT (SEQ ID No: 5) |
| rs1800469/TGEb1(-509)R | AGGCTGGGAAACAAGGTAGGAG (SEQ ID No: 6) |
| rs2241715/TGEb1(intron)F | CAATCCTCTTCTCCCCAACA (SEQ ID No: 7) |
| rs2241715/TGEb1(intron)R | TACTCAGCAAACCCCAAAGG (SEQ ID No: 8) |
| rs4253527/SPA1(R219W)F | CCTGGAGACTTCCGCTACTCAG (SEQ ID No: 9) |
| rs4253527/SPA1(R219W)R | GATGGTCAGTCGGGAGTACAGG (SEQ ID No: 10) |
| ASPE primers | |
| rs1800469/TGFb1(-509)T: 12* | TACACTTTCTTTCTTTCTTTCTTTGCCTCCTGACCCTTCCATCCT (SEQ ID No: 11) |
| rs1800469/TGFb1(-509)C: 94* | CTTTCTATCTTTCTACTCAATAATGCCTCCTGACCCTTCCATCCC (SEQ ID No: 12) |
| rs2241715/TGFb1(intron)C: 6* | CTTTTACAATACTTCAATACAATCAGACAGACCTCCCGCCCTGGGAGAG (SEQ ID No: 13) |
| rs2241715/TGFb1(intron)G: 20* | CTTTTACAATACTTCAATACAATCAGACAGACCTCCCGCCCTGGGAGAG (SEQ ID No: 14) |
| rs1800451/MBL57G: 59* | TCATCAATCAATCTTTTTCACTTTACCTGGTTCCCCCTTTTCTC (SEQ ID No: 15) |
| rs1800451/MBL57A: 30* | TTACCTTTATACCTTTCTTTTTACACCTGGTTCCCCCTTTTCTT (SEQ ID No: 16) |
| rs5030737/MBL52C: 28* | CTACAAACAAACAAACATTATCAACTTCCCAGGCAAAGATGGGC (SEQ ID No: 17) |
| rs5030737/MBL52T: 46* | TACATCAACAATTCATTCAATACACTTCCCAGGCAAAGATGGGT (SEQ ID No: 18) |
| rs1800450/MBL54C: 12* | TACACTTTCTTTCTTTCTTTCTTTTTCCCCCTTTTCTYCCTTGGTGC (SEQ ID No: 19) |
| rs1800450/MBL54T: 37* | CTTTTCATCTTTTCATCTTTCAATTTCCCCCTTTTCTYCCTTGGTGT (SEQ ID No: 20) |
| rs7096206/MBL_XY_C: 30* | TTACCTTTATACCTTTCTTTTTACCCATTTCTTCTCACTGCCACC (SEQ ID No: 21) |
| rs7096206/MBL_XY_G: 59* | TCATCAATCAATCTTTTTCACTTTCCATTTCTTCTCACTGCCACG (SEQ ID No: 22) |
| rs4253527/SPA1R219W_A: 10 | ATCATACATACATACAAATCTACACACACACTGCTCTTTTCCCCA (SEQ ID No: 23) |
| rs4253527G/SPA1R219W: 8 | AATCCTTTTACATTCATTACTTACCACACACTGCTCTTTTCCCCG (SEQ ID No: 24) |

*Please note:
1. First 21 bp correspond to an anti-Zip sequence of code indicated by the last two digits in the end of ASPE primer name.
2. For tagging the ASPE primers TagIT ™ program (TmBioscience) was used: https://tagit.luminexcorp.com/tagit/welcomejsp.
3. All ASPE reactions were done in separate multiplexed to avoid primer-dimer formation, therefore the same codes for Zip tags were used.

### EXAMPLE 3

### Measuring of MBL2 Levels in Plasma

The MBL2 LiquiChip assay was developed in house based on the 131-01 monoclonal antibodies (AntibodyShop). Since active MBL2 is only capable of binding Mannose in multimeric form and all missense mutations in the MBL2 gene abolish the multimerization of the mutant MBL2 protein, a bead based assay was developed that utilizes the same antibody for binding (131-01) and detection (131-01B, biotin modified). To couple the LiquiChip Activated Beads (Qiagen, Cat. No: 922543) to the antibodies the bead stock was vortexed for 30 sec at full speed and another 30 min in the dark to completely resuspend the beads. 100 μl of LiquiChip Activated Beads suspension was pipetted into a 1.5 ml siliconized polypropylene copolymer reaction microtubes (Fisher Scientific, cat. no. 3544350). The antibody was diluted in coupling buffer (50 mM MES, pH 6.5) to a concentration of 0.2 mg/ml in a volume of 50 μl (10 μg protein) and was added to the beads. The beads were incubated for 2 hr in the dark at room temperature with shaking The bead suspension was centrifuged for 3 min at 10,000×g and the supernatant was removed stepwise in small aliquots to minimize bead loss. Beads were washed by resuspension in 500 μl of PBS and after centrifugation for 3 min at 10,000×g the supernatant was discarded. The beads were stored in 100 μl PBS/1% BSA at a concentration 1.25×10⁵ beads/ml at 4° C. in the dark. Plasma samples were cleared by centrifugation for 5 min at full speed, the supernatant was additionally filtered through a Millipore MultiScreen filter plate by centrifugation at 3500 rpm for 5 min. The samples were diluted at least 1:2, e.g. 50 μl plasma plus 100 μl of LiquiChip Human Serum Dilution Buffer (Qiagen, Cat No: 922300) and loaded on the Millipore MultiScreen filter plates. Next 20 μl beads mixture (diluted 1/20 in PBS/BSA, equivalent to 1250 beads per well) was added per well, mixed for 10 sec on a microplate shaker at 850 rpm and incubated for 2 hours in the dark at room temperature while shaking The beads were washed twice—drained on vacuum, resuspended in 150 μl PBS-TW, vortexed and vacuumed again. The biotinylated secondary antibodies were diluted in PBS-TW (1:5000 for 131-01B) and 100 μl of diluted antibodies was added to each well and incubated for 1.5 hours in the dark at room temperature on a microplate shaker at 850 rpm. The reactions were developed with addition of 100 ng Streptavidin-R-PE in a volume of 10 μl, incubated for 30 min in the dark. The reactions were analyzed on Luminex¹⁰⁰ LiquiChip Reader, according to manufacturer instructions. Minimum of 100 beads were counted for each analyte and Mean Fluorescence Index (MFI) values were calculated by the software.

A similar method was conducted to determine SP-A1 levels in plasma using immunized rabbit serum.

All relevant portion of references referred to herein are incorporated herein by reference.

We claim:

1. A method of diagnosing risk of pulmonary disease in a mammal with cystic fibrosis comprising:
screening in a biological sample obtained from the mammal for at least one of a deficient MBL2 or SP-A1 gene mutant, and an over-expressing TGFB1 gene, wherein identification of at least one of a deficient MBL2 or SP-A1 gene mutant, and an over-expressing TGFB1 gene is indicative of a risk of pulmonary disease.

2. A method as defined in claim 1, wherein the MBL2 gene mutant is an under-expressing mutant.

3. A method as defined in claim 1, wherein the MBL2 gene mutant includes a mutation at at least one position in the gene selected from the group consisting of position 154, 161, 170 and −221.

4. A method as defined in claim 1, wherein the SP-A1 gene mutant yields a dysfunctional protein.

5. A method as defined in claim 1, wherein the SP-A1 gene mutant yields an R219W SP-A1 mutant.

6. A method as defined in claim 1, wherein the SP-A1 gene mutant includes a missense mutation which disrupts a Gly-X-Y repeat of SP-A1.

7. A method as defined in claim 1, wherein the TGFB1 gene comprises a mutation as set out in rs 2241715.

8. A method as defined in claim 1, wherein the TGFB1 gene comprises a mutation that yields an L10P mutated TGFB1.

9. A method of diagnosing risk of pulmonary disease in a mammal having cystic fibrosis comprising:
determining in a biological sample obtained from the mammal the levels of at least one of MBL2 or SP-A1, and the level of TGFB1, wherein a determination of MBL2 or SP-A1 deficiency, and TGFB1 over-expression in comparison to control levels in a healthy mammal is indicative of a risk of pulmonary disease.

10. A method as defined in claim 9, wherein the level of MBL2 expression is determined.

11. A method as defined in claim 10, wherein MBL2 is determined to be expressed at a level of less than about 500 μg/L.

12. A method as defined in claim 9, wherein SP-A1 expression level is determined.

13. A method as defined in claim 9, wherein TGFB1 is determined to be expressed at a level greater than about 5 μg/L.

14. A method of treating a mammal at risk of developing pulmonary disease comprising inhibiting the expression of TGFB1 in said mammal.

15. A method as defined in claim 14, including the additional step of administering a therapeutically effective amount of MBL2 or SP-A1.

16. A method as defined in claim 15, wherein the therapeutically effective amount is sufficient to render an MBL2 or SP-A1 blood level of at least about 500 μg/L.

17. A method as defined in claim 14, including the additional step of administering an antibiotic effective to treat lung disease.

18. A method as defined in claim 17, wherein the antibiotic is selected from the group consisting of TOBY, azythromycin, Dornase Alfa, Denufosol and hypertonic saline in nebulized form.
